(19) United States
(12) Patent Application Publication
(10) Pub. No.: US 2018/0164264 A1
(43) Pub. Date: Jun. 14, 2018

(54) FILTER FOR VISUAL GAS SENSOR

(71) Applicant: Jesse Jake Savage, Boca Raton, FL (US)

(72) Inventor: Jesse Jake Savage, Boca Raton, FL (US)

(21) Appl. No.: 15/375,163

(22) Filed: Dec. 11, 2016

(51) Int. Cl. G01N 31/22; G01N 33/00

(52) U.S. Cl. CPC G01N 31/224; G01N 33/0004

(57) ABSTRACT

a gas detecting device has a gas detecting region on a detection card. A window extends over the detecting region, protecting it from ultraviolet light. The window includes air inlets that allow air to travel underneath the window and interact with the detecting region. Filters block or remove any contaminants in the air prior to the air interacting with the detecting region. As a result, false positives are minimized and the functional lifespan of the detecting device is extended.

### STATEMENT REGARDING FEDERALLY SPONSORED RESEARCH OR DEVELOPMENT

Not Applicable.

### THE NAMES OF PARTIES TO A JOINT RESEARCH AGREEMENT

Not Applicable

### INCORPORATION-BY-REFERENCE OF THE MATERIAL SUBMITTED ON A COMPACT DISC

Not Applicable.

### COPYRIGHT NOTICE

Not Applicable

### BACKGROUND OF THE INVENTION

### Field of the Invention

The present invention relates to a gas sensor. More particularly, the invention relates to a gas sensor having a protective casing for the sensing mechanism.

### Description of the Related Art

Carbon monoxide is a colorless, odorless and tasteless gas that is slightly less dense than air. It is a competitive inhibitor of hemoglobin and therefore toxic to humans and most animals. It is produced by internal combustion engines and it is therefore important to monitor for the presence of carbon monoxide in most vehicles, such as for example automobiles, airplanes, trucks, ships and the like.

Many relatively simple devices have been developed to detect the presence of carbon monoxide. One commonly used device is a card having a substance that changes color in the presence of carbon monoxide. These carbon monoxide sensors, commonly called a “spot” (or “patch”) sensor (examples—ASA, Sportys, ProLabs, InspectUSA and many other manufacturers/brands) include a region on the card where powdered silica (sand-like material) is glued, fused and/or melted together in a thin layer and typically spread out into a 0.5 inch circle. When exposed to carbon monoxide, the substance changes color. This type of visual gas sensor has been commercially used since the 1970's.

The simplicity and low cost of these spot sensors have made them a favorite among airplane pilots to detect carbon monoxide in their cockpits. Once the sensor is removed from its air tight packaging, these sensors are completely susceptible to all of the elements in the environment.

Spot sensors have had this problem since their introduction in the 1970's. The accuracy or longevity of the sensors has not been improved by the sensor manufacturers, aftermarket parts manufacturers or end users. These sensors' life limiting factors include: vulnerability to—sunlight/UV, large air particles, pollutants, humidity, liquids, aerosols, tampering, touching, abuse, and other contaminants and physical disruptions of the sensing component(s). These life limiting factors drastically reducing their effectiveness and their useful operational life.

FIG. 1 shows a typical carbon monoxide sensor 10 of the prior art. The sensor 10 consists of a planar card 12 with a detection region 14. As is typical in the prior art, the detection region 14 is circular and is approximately ½ inch in diameter. The detection region 14 of an unused sensor is typically tan in color. Upon exposure to carbon monoxide, it becomes gray and eventually black. However, over a few months, the color of the detection region 14 changes regardless of exposure to carbon monoxide. This is due to a variety of factors including exposure to light and contaminants unrelated to carbon monoxide.

These spot sensors typically have a 3 year shelf life in their airtight packaging. Once removed from the airtight packaging, the sensors typically only last 1 to 3 months, depending on the amount of life limiting factors, listed above, in their operational environment, before becoming permanently discolored and useless.

The above-described deficiencies of today's systems are merely intended to provide an overview of some of the problems of conventional systems, and are not intended to be exhaustive. Other problems with the state of the art and corresponding benefits of some of the various non-limiting embodiments may become further apparent upon review of the following detailed description.

In view of the foregoing, it is desirable to provide spot sensors having an extended useful lifespan.

### BRIEF SUMMARY OF THE INVENTION

Disclosed is a visual gas sensor having an extended useful operational life.

A carbon monoxide sensor in accordance with principles of the invention includes a planar detection card having a detection region that changes color when exposed to carbon monoxide. A raised window extends over the detection region and defines an air pocket between the window and the detection region. The window has a transparent region over the detection region. A plurality of inlets in the window provide fluid communication between the air pocket and the exterior of the window. A molecular air filter within the air pocket covers the plurality of inlets in the window without covering the detection region. A hydrophobic filter within the air pocket covers the molecular air filter without covering the detection region.

It is therefore an object of the present invention to provide a sensor having a protective window and filters over the detector to prevent contaminants from interacting with the detector.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### BRIEF DESCRIPTION OF THE SEVERAL VIEWS OF THE DRAWINGS

A more complete understanding of the present invention, and the attendant advantages and features thereof, will be more readily understood by reference to the following detailed description when considered in conjunction with the accompanying drawings wherein:

FIG. 1 is a perspective view of a detection device of the prior art;

FIG. 2 is a perspective view of a detection device in accordance with the principles of the invention;

FIG. 3 is an exploded perspective view of a detection device in accordance with the principles of the invention;

FIG. 4 is a perspective view of a window of a detection device in accordance with principles of the invention;

FIG. 5 is a cross-sectional view of a window of a detection device in accordance with principles of the invention;

FIG. 6 is a perspective view of a molecular air filter of a detection device in accordance with principles of the invention;

FIG. 7 is a perspective view of a hydrophobic filter of a detection device in accordance with the principles of the invention;

FIG. 8 is a perspective cutaway view of a detection device in accordance with the principles of the invention;

FIG. 9 is a side cutaway view of a detection device in accordance with the principles of the invention;

FIG. 10 is a top plan view of an alternative embodiment of a detection device in accordance with principles of the invention;

FIG. 11 is a cross-sectional view of an alternative embodiment of a detection device in accordance with principles of the invention.

### DETAILED DESCRIPTION OF THE INVENTION

The invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The disclosed subject matter is described with reference to the drawings, wherein like reference numerals are used to refer to like elements throughout. In the following description, for purposes of explanation, numerous specific details are set forth in order to provide a thorough understanding of the various embodiments of the subject disclosure. It may be evident, however, that the disclosed subject matter may be practiced without these specific details. In other instances, well-known structures and devices are shown in block diagram form in order to facilitate describing the various embodiments herein.

As used herein, “contaminant[s]” is generally used to refer to particulate matter, colloids, gases and other physical material that, upon exposure to a detecting region of a sensor, shortens the operational lifetime of the sensor.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

Disclosed is a gas detecting device in accordance with the principles of the invention. The device includes a detection region that changes color when exposed to a specific airborne material, such as for example carbon monoxide. A relatively flat, cylindrical or dome-shaped window is sealed to the detection card over the detection region. The window includes several inlets through which ambient air may pass and come into contact with the detection region. One or more filters are placed over these inlets to block undesirable contaminants that may cause the detection region to change color not in response to the gas being detected.

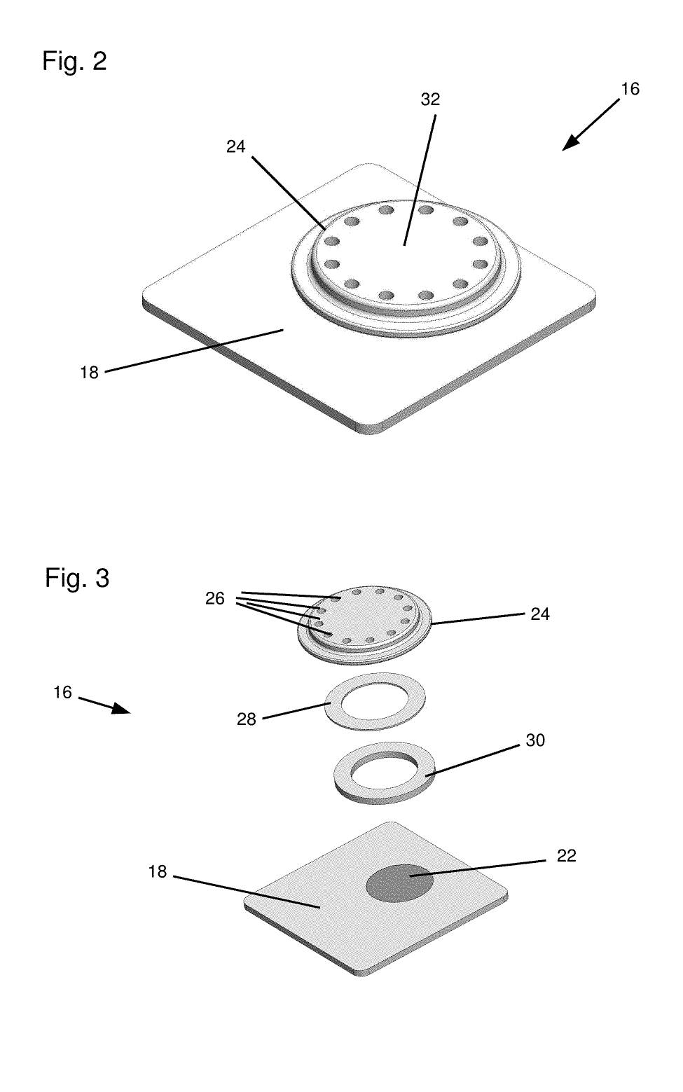

FIGS. 2 and 3 show a sensor 16 in accordance with the principles of the invention. The sensor 16 includes a raised window 24 affixed to the detection card 18. The window 24 has a top surface 32 that extends over the detection region 22. The window 24 is transparent in the visible wavelengths, allowing the detection region 22 to be viewed through it.

The detection region 22 of this embodiment is a layer of material embedded in the detection card 18 that changes color upon exposure to a specific gas, in this case carbon monoxide. Optionally, the detection region can be formed by a cavity filled with a detection material. Optionally, the detection region can be formed from a small cup or container having an open top and a bottom that is affixed to the detection card.

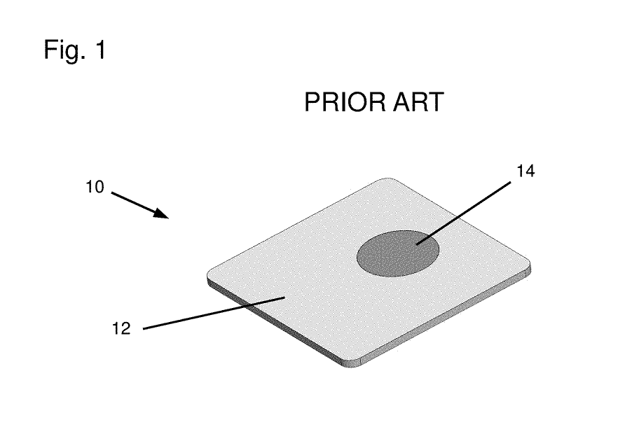

FIG. 3 shows an exploded view of sensor 16 in accordance with the principles of the invention. The sensor 16 includes a planar detection card 18 having a detection region 22 similar to the sensor 10 shown in FIG. 1. The sensor 16 also includes a cylindrical window 24 having a plurality of inlets 26 forming a ring about the top of the window 24.

This embodiment includes a molecular air filter 28 and a hydrophobic filter 30, both configured as annular rings that aligns with the inlets 26 of the window 24. The filters 28 and 30 are configured such that they do not overlap or partially obscure the detection region 22. They are also configured to completely cover the air inlets 26, thereby preventing contaminants from contacting the detection region.

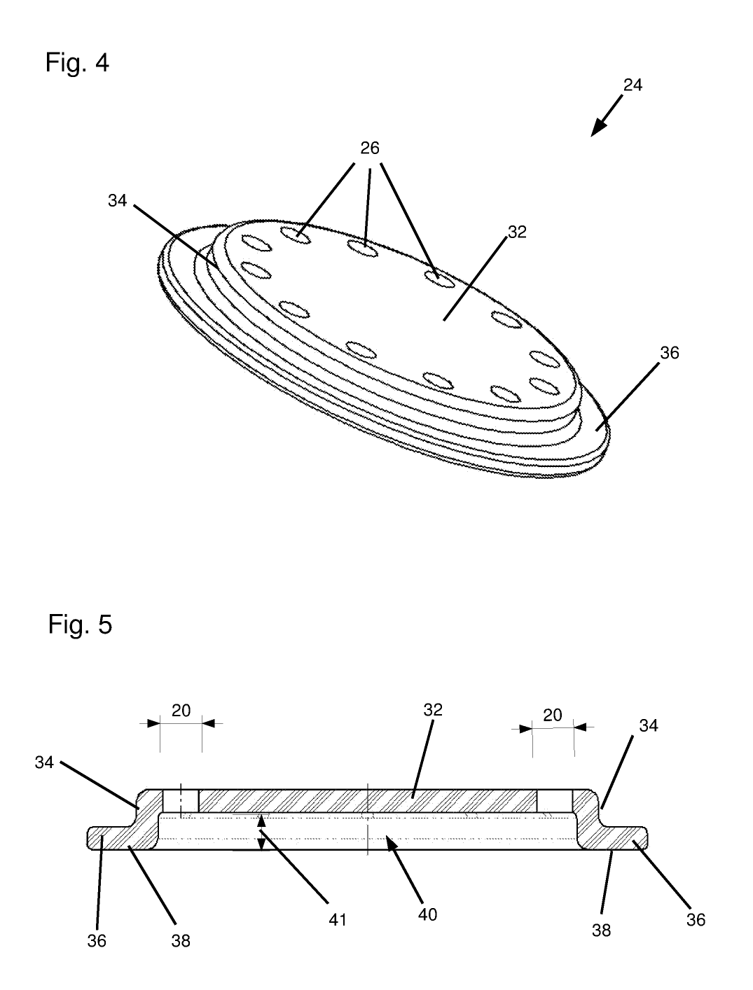

FIGS. 4 and 5 show the window 24 in more detail. The window 24 of this embodiment includes a disk-shaped top 32, a peripheral wall 34 and a flange 36. The bottom side 38 of the flange 36 is affixed to the planar detection card 18 by adhesive, glue, double sided tape, epoxy, physical encapsulation, plastic welding, ultrasonic welding or other methods known in the art, including combinations of those listed. The top 32 and wall 34 defined an air pocket 40 between the window 24 and the detection card 18.

The window 24 of this embodiment is made of a transparent material that filters ultraviolet light. Those skilled in the art will appreciate that there are a wide variety of materials that are transparent to visible wavelengths but filter other wavelengths such as ultraviolet light. Because the window is transparent, the detection region is viewable through it. The window 24 prevents physical damage, tampering, touching or abuse of the detecting region of the device. Optionally, the peripheral wall 34, flange 36 and airflow region 20 may not be transparent because it is not necessary to view objects underneath these regions of the window 24.

The top 32 of the window 24 is continuous, planar and a constant distance 41 from the detection region 22 over which it extends. It is generally preferable that at least the top 32 of the window is rigid or semi-rigid in order to prevent direct contact with the detection region 22. The air inlets 26 are arranged on the top 32 of the window 24 in an airflow region 20 that substantially aligns with the filters as explained in more detail below. In this embodiment, the airflow region 20 is configured as an annular ring adjacent to the peripheral wall 34. The inlets 26 of the airflow region 20 provide fluid communication between the air pocket 40 and the surrounding air. The inlets 26 thus allow ambient air to flow into the air pocket 40 formed by the window 24 over the detection region.

The air pocket 40 is defined by the top 32, the peripheral wall 34 and the detection card 18. The air pocket 40 has a height 41 defined by the distance between the top 32 of the window 24 and the bottom 38 of the flange 36. In this embodiment, this height is constant. The bottom 38 of the annular flange 36 is flush with the detection card 18. Therefore, the height 41 is equally defined as the distance from the top 32 to the detection region 22 it extends over. The area of the air pocket 40 is larger than the area of the detection region 22 so that it may accommodate the filters 28 and 30 which surround but do not obstruct the detection region. In this embodiment, the width of the air pocket is equal to the internal diameter of the cylindrical peripheral 34 wall and also equal to the width of the detection region 22 plus twice the width of the filters 28 and 30. No ambient air enters the air pocket 40 and interacts with the detection region 22 without first passing through an air inlets 26. In this embodiment, the air inlets 26 are circular. The air inlets 26 may optionally have a rectangular, square, polygonal, ellipsoid or other configuration. The air inlets may also optionally be located on the peripheral wall 34. In general, the airflow region and the air inlets with an the airflow region should be configured to optimize airflow without compromising the integrity of the air pocket. The airflow region and air inlets should generally also be configured to accommodate secure positioning of one or more air filters that completely cover the inlets.

FIGS. 4 and 5 show an exemplary window 24 having a circular configuration. The top 32 is circular, the airflow region 20 is annular, the peripheral wall 34 is cylindrical and the flange 36 is annular. The invention is not limited to a circular configuration, but other geometries may be used.

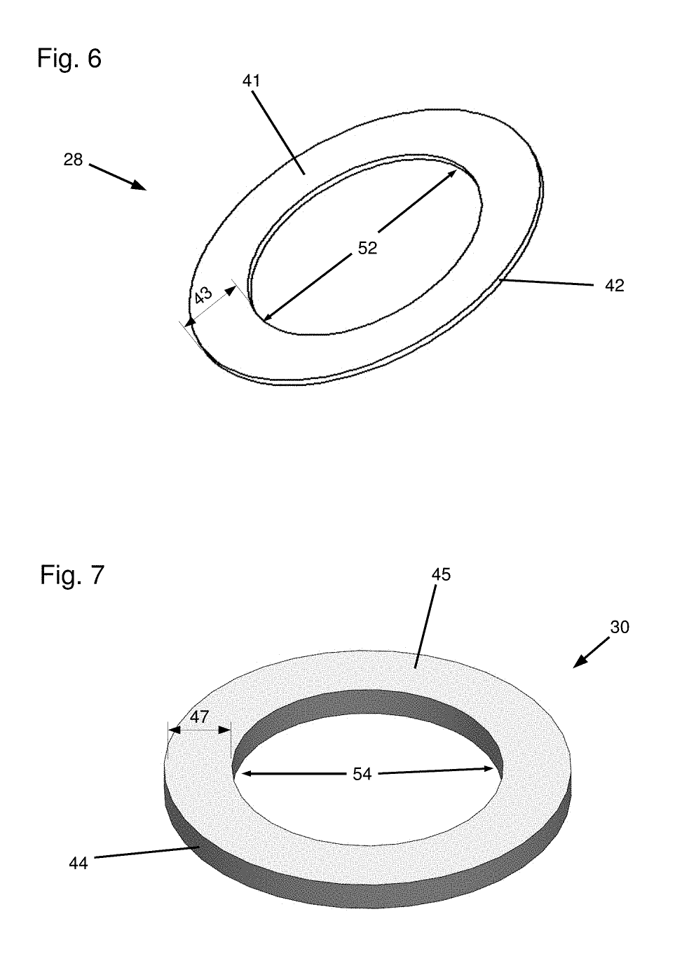

FIG. 6 shows a molecular air filter 28 in accordance with the principles of the invention. In this embodiments, air filter 28 has an annular, ring-shaped body 41 defined by a thickness 42 and a width 43. The internal diameter 52 of the molecular air filter 28 is equal to or greater than the diameter of the detection region 22. The internal diameter 52 plus twice the width 43 is equal to the internal diameter of the cylindrical peripheral wall 34.

The molecular air filter 28 limits what particles may pass through it. When the sensor 16 is fully assembled, the molecular air filter 28 is compression fit flush against the air inlets 26. In this embodiment, the molecular air filter 28 is made of borosilicate glass. Optionally, other materials capable of limiting what particles pass through them may also be suitable.

FIG. 7 shows a hydrophobic filter 30 in accordance with the principles of the invention. The hydrophobic filter 30 is configured as an annular ring and has a top side 45, a width 47, a thickness 44 and an internal diameter 54. The width 47 is equal or greater than to the width 43 of the molecular air filter 28 of FIG. 6. The internal diameter 54 is equal to or greater than the diameter of the detection region 22. The hydrophobic filter 30 limits the size of liquid particles passing through it from the air inlets 26 and into the air pocket 40. In this embodiment, the hydrophobic filter 30 is comprised of a foam having 100+ pores per inch. The hydrophobic filter 30 generally prevents moisture from interacting with the detection region of a gas detecting device. Depending on the operating environment and the gas sensor's specifications, additional filtering components can be added, removed, combined or modified to provide optimal gas sensor performance and longevity. The top side 45 is configured to lie flush against the molecular air filter 28. In this embodiment, the top side 45 is substantially planar. Optionally, the top side 45 may be angled include ridges and channels or grooves, or have other structural features that facilitate a flush, continuous engagement with the molecular air filter 28 positioned above it.

The hydrophobic filter 30 is compressible. The molecular air filter 28 may or may not also be compressible. Optionally, the hydrophobic filter 30 may not be compressible and instead only the molecular air filter 28 is compressible.

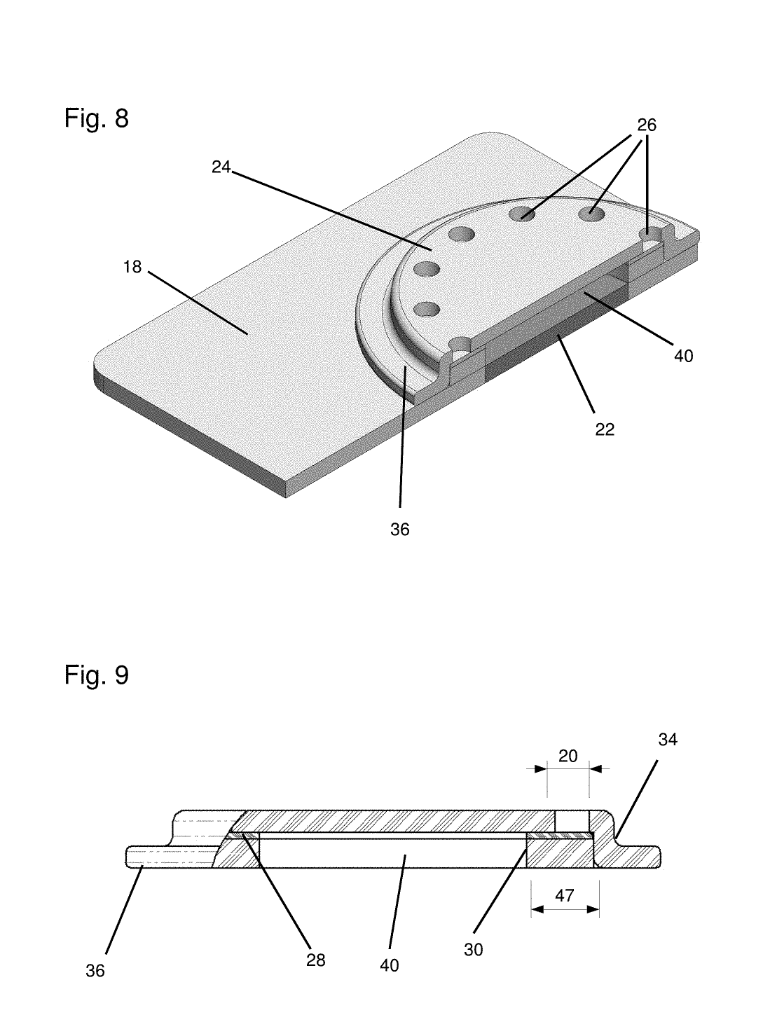

FIGS. 8 and 9 show the sensor 16 fully assembled. The combined thicknesses of the molecular air filter 28 and the hydrophobic filter 30 is greater than the height of the air pocket 40. As a result, the filters are firmly held in place flush against each other and the inlets 26. By forming a compression fit between the detection card 18 and the airflow region 20 of the window 24, the construction of the device ensures that any particles traveling into the air pocket 40 must first pass through both filters. The molecular air filter and the hydrophobic filter have widths 43 and 47 that extend beyond the airflow region 20 to prevent incoming particles from traveling only through the molecular air filter 28 without passing through the hydrophobic filter 30. That is, the bottom filter, in this case the hydrophobic filter, is substantially thicker than the molecular air filter. Both filters have widths that cause them to extend further medially inward from the cylindrical wall 34 than the airflow region 20. Both filters surround the detection region 22, but do not interfere with the functionality or visibility of the detection region.

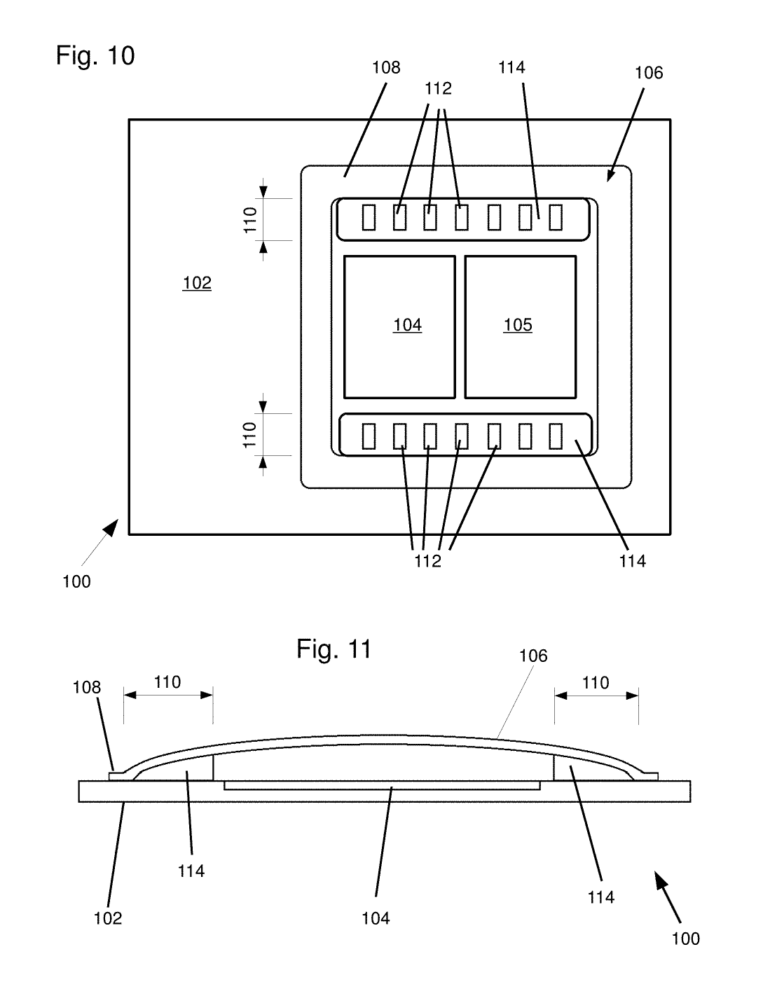

FIGS. 10 and 11 show an alternative embodiment of a gas sensor 100. The gas sensor 100 has a planar detection card 102 with two rectangular gas detecting regions 104 and 105. Each of the two detecting region's 104 and 105 change color upon exposure to different gases. A window 106 is affixed to the planar detection card 102 along a rectangular flange 108 that lies flush against the detection card 102. The window 106 of this embodiment is dome-shaped and extends over the detecting regions 104 and 105. As a result, the distance between the window 106 and the detection card 102 is not constant.

The window 106 has two airflow regions 110, each having a plurality of rectangular inlets 112. Each airflow region 110 has a corresponding filter 114 aligned with it and located underneath the window 106. In this embodiment, only one type of filter 114 is used. The air filters 114 of this embodiment are secured in place flush against the inlets 112 by an adhesive applied between the filter 114 and the window 106 and/or the detection card 102.

In normal operating environments, no scheduled maintenance of the air filtering device is required. In very dusty operating environments, the air inlet holes may become clogged but can be easily cleared by blowing air on them.

In use, the detection card is placed in a region to be monitored. The filter(s) covering the air inlets prevents water and contaminants from interacting with the detection region. The window blocks ultraviolet light and prevents it from interacting with the detection region. As a result, the functional lifespan of the detection card is greatly increased to several months or more. In the above description, the gas being detected is generally referred to as carbon monoxide. This is only intended as an example, and the invention may be practiced when detecting other gases, colloids or fine particulates in ambient air. For example, the detecting region, instead of detecting carbon monoxide, may function to detect carbon dioxide, chlorofluorocarbon's, smoke, other exhaust fumes or other substances.

Whereas, the present invention has been described in relation to the drawings attached hereto, it should be understood that other and further modifications, apart from those shown or suggested herein, may be made within the spirit and scope of this invention. Descriptions of the embodiments shown in the drawings should not be construed as limiting or defining the ordinary and plain meanings of the terms of the claims unless such is explicitly indicated.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

1. A sensor comprising:
a planar detection card having a detection region that changes color when exposed to a material to be detected;
a raised window extending over the detection region and defining an air pocket between the window and the detection region;
an airflow region of the window having a plurality of air inlets in the window that provide fluid communication between the air pocket and the exterior of the window; and,
one or more filters covering the plurality of air inlets in the window and not covering the detection region;
wherein the filter allows passage of the material to be detected but blocks passage of other material.

2. The sensor of claim 1 wherein the window comprises:
a planar circular top;
a cylindrical peripheral wall; and;
an annular flange permanently affixed to the detection card;
wherein, the airflow region is configured as an annular ring on the circular top and adjacent to the cylindrical peripheral wall.

3. The sensor of claim 2 wherein the filter comprises:
an annular molecular air filter that limits the size of particles entering the air pocket; and,
an annular hydrophobic filter that prevents moisture from entering the air pocket;
wherein the annular molecular air filter is positioned on top of the annular hydrophobic filter and both filters are held in place by a compression fit between the window and the detection card.

4. The sensor of claim 3 wherein:
the air pocket has a height defined by the window and the detection card;
the molecular air filter has a thickness;
the hydrophobic filter has a thickness and is compressible; and,
the combined thickness of the molecular air filter and the hydrophobic filter is greater than the height of the air pocket such that when the sensor is fully assembled the molecular air filter and hydrophobic filter are secured in place within the air pocket by a compression fit.

5. The sensor of claim 4 wherein the window is transparent to visible wavelengths and filters ultraviolet light.

6. The sensor of claim 5 wherein the detection region detects the presence of carbon monoxide.

7. The sensor of claim 6 wherein the window is comprised of a rigid material.

8. The sensor of claim 7 wherein the window is transparent to visible wavelengths only across a region directly above the detection region of the detection card.

9. The sensor of claim 1 wherein the window comprises:
a rectangular, dome-shaped top; and,
a rectangular flange affixed to the detection card.

10. The sensor of claim 9 wherein:
the airflow region comprises two rectangular regions on opposing sides of the window; and,
the one or more filter comprise two filters, one underneath each of the airflow regions.

11. The sensor of claim 10 wherein the filters are secured in position by an adhesive.

12. The sensor of claim 11 wherein the detection region detects the presence of carbon monoxide.

13. A method of detecting an airborne material comprising:
providing a sensor comprising:
a planar detection card having a detection region that changes color when exposed to a material to be detected;
a raised window extending over the detection region and defining an air pocket between the window and the detection region;
an airflow region of the window having a plurality of air inlets in the window that provide fluid communication between the air pocket and the exterior of the window; and,
one or more filters covering the plurality of air inlets in the window and not covering the detection region;
wherein the filter allows passage of the material to be detected but blocks passage of other material;
placing the sensor in a region where the material to be detected may be present;
monitoring the sensor over a period of time for a change in color of the detection region.

14. The method of detecting an airborne material of claim 13 wherein the window of the sensor comprises a planar circular top, a cylindrical peripheral wall, and an annular flange permanently affixed to the detection card;
wherein, the airflow region is configured as an annular ring on the circular top and adjacent to the cylindrical peripheral wall.

15. The method of detecting an airborne material of claim 14 wherein the filter of the sensor comprises an annular molecular air filter that limits the size of particles entering the air pocket and an annular hydrophobic filter that prevents moisture from entering the air pocket;
wherein the annular molecular air filter is positioned on top of the annular hydrophobic filter and both filters are held in place by a compression fit between the window and the detection card.

16. The method of detecting an airborne material of claim 15 wherein:
the air pocket has a height defined by the window and the detection card;
the molecular air filter has a thickness;
the hydrophobic filter has a thickness and is compressible; and,
the combined thickness of the molecular air filter and the hydrophobic filter is greater than the height of the air pocket such that when the sensor is fully assembled the molecular air filter and hydrophobic filter are secured in place within the air pocket by a compression fit.

17. The method of detecting an airborne material of claim 16 wherein the window of the sensor is transparent to visible wavelengths and filters ultraviolet light.

18. The method of detecting an airborne material of claim 17 wherein the detection region of the sensor detects the presence of carbon monoxide.

19. The method of detecting an airborne material of claim 18 wherein the window of the sensor is comprised of a rigid material.

20. The method of detecting an airborne material of claim 19 wherein the window of the sensor is transparent to visible wavelengths only across a region directly above the detection region of the detection card.
